(19) United States
(12) Patent Application Publication
(10) Pub. No.: US 2025/0195012 A1
(43) Pub. Date: Jun. 19, 2025

(54) RECONSTRUCTION METHOD OF ARTERIAL BLOOD PRESSURE

(71) Applicant: Industry-Academic Cooperation Foundation, Chosun University, Gwangju (KR)

(72) Inventors: Jae Hyo JUNG, Gwangju (KR); Geng Jia ZHANG, Gwangju (KR)

(73) Assignee: Industry-Academic Cooperation Foundation, Chosun University, Gwangju (KR)

(21) Appl. No.: 18/774,040

(22) Filed: Jul. 16, 2024

(30) Foreign Application Priority Data
Dec. 15, 2023 (KR) 10-2023-0183119

(51) Int. Cl. A61B 5/00; A61B 5/352

(52) U.S. Cl. CPC A61B 5/7278; A61B 5/352; A61B 5/7203; A61B 5/725; A61B 5/7267

(57) ABSTRACT

The present disclosure relates to a method of reconstructing an arterial blood pressure (ABP) signal corresponding to the morphological feature of a combination signal of ECG and PPG on the basis of the morphological feature. The method of reconstructing an ABP signal according to an embodiment of the present disclosure includes: generating an ECG-PPG signal by combining ECG and PPG signals corresponding to an ABP signal; generating a plurality of ECG-PPG signals for learning by applying a plurality of noises, which is different in intensity, to the ECG-PPG signal; training a neural network model to receive the ECG-PPG signals for learning and output the ABP signal; and reconstructing an ABP signal of a target user by inputting an ECG-PPG signal of the target user into the neural network model.

### CROSS REFERENCE TO RELATED APPLICATION

The present application claims priority to Korean Patent Applications No. 10-2023-0183119, filed Dec. 15, 2023, the entire contents of which are incorporated herein for all purposes by this reference.

### BACKGROUND

### Technical Field

The present disclosure relates to a method of reconstructing an arterial blood pressure (ABP) signal corresponding to the morphological feature of a combination signal of ECG and PPG on the basis of the morphological feature.

### Description of the Related Art

High blood pressure is the reason for various cardiovascular diseases and cerebrovascular diseases such as a stroke, myocardial infarction, and heart failure, and according to the statistical data on cause of death of 2022 by the National Statistical Office of Korea, it was reported that four of the top 10 of causes of death are cardiocerebrovascular diseases (cardiovascular diseases, cerebrovascular diseases, diabetes, and hypertensive diseases).

Since cardiocerebrovascular diseases are usually generated by interception of blood flow due to stricture of blood vessels, it is very important to monitor blood pressure in order to prevent diseases, and for this purpose, recently, various smart devices and medical applications that obtain photoplethysmography (PPG) and electrocardiogram (ECG) signals and determine a biomarker relating to a blood pressure are being developed.

However, biomarkers that can be obtained a PPG signal or an ECG signal are limitative more than biomarkers that can be obtained from an ABP signal, so it is more important to determine an ABP waveform in terms of clinic, but there is a limitation that it is very difficult to measure a clear ABP signal in the medical field.

### SUMMARY

An objective of the present disclosure is to estimate an ABP signal or further estimate a blood pressure from a signal obtained by combining ECG and PPG using a neural network model.

The objectives of the present disclosure are not limited to those described above and other objectives and advantages not stated herein may be understood through the following description and may be clear by embodiments of the present disclosure. Further, it would be easily known advantages of the present that the objectives disclosure may be achieved by the configurations described in claims and combinations thereof.

In order to achieve the objectives described above, a method of reconstructing an arterial blood pressure signal according to an embodiment of the present disclosure includes: generating an ECG-PPG signal by combining ECG and PPG signals corresponding to an ABP signal; generating a plurality of ECG-PPG signals for learning by applying a plurality of noises, which is different in intensity, to training a neural network model to the ECG-PPG signal; receive the ECG-PPG signals for learning and output the ABP signal; and reconstructing an ABP signal of a target user by inputting an ECG-PPG signal of the target user into the neural network model.

In an embodiment, the generating of an ECG-PPG signal includes: removing noises in the ECG signal and the PPG signal by passing the ECG signal and the PPG signal through a band pass filter (BPF); and generating the ECG-PPG signal by combining the ECG signal and PPG signal with the noises removed.

In an embodiment, the generating of an ECG-PPG signal includes: detecting an R peak in the ECG signal; detecting a maximum peak in the PPG signal; aligning the ECG signal and the PPG signal on the basis of a time offset between the R peak and the maximum peak; and generating the ECG-PPG signal by combining the aligned ECG signal and PPG signal.

In an embodiment, the generating of an ECG-PPG signal includes: detecting a plurality of R peaks in an ECG signal in a unit time period; detecting a plurality of maximum peaks in a PPG signal in the unit time period; computing a time offset between an R peak and a maximum peak of a preset order of the plurality of R peaks and the plurality of maximum peaks; aligning the ECG signal and the PPG signal by applying the time offset to the ECG signal or the PPG signal; and generating the ECG-PPG signal by combining the aligned ECG signal and PPG signal.

In an embodiment, the generating of an ECG-PPG signal includes generating the ECG-PPG signal by connecting the ECG signal and the PPG signal such that the ECG signal and the PPG signal are temporally continuous.

In an embodiment, the generating of an ECG-PPG signal for learning includes generating a plurality of ECG-PPG signals for learning by applying each of noises having a plurality of preset Signal-to-Noise Ratios (SNRs) to the ECG-PPG signal.

In an embodiment, the SNR is defined by the following [Equation 1],

$$S N R = 10 {log}_{10} \frac{Raw\_signal}{P noise} \qquad [ Equation 1 ]$$

(where Raw_signal is average intensity of the ECG-PPG signal and P_noise is the noise).

In an embodiment, the training of a neural network model includes applying supervised learning to the neural network model by setting each of the ECG-PPG signals as input data of the neural network model and setting the APB signal as output data of the neural network model.

In an embodiment, the method further includes computing a systolic blood pressure and a diastolic blood pressure from the APB signal, wherein the training of a neural network model includes training the neural network model such that the neural network model receives each of the ECG-PPG signals and outputs the ABP signal and the systolic and diastolic blood pressures, and the reconstructing of an ABP signal includes determining an ABP signal and systolic and diastolic blood pressures of a target user by inputting an ECG-PPG signal of the user into the neural network model.

In an embodiment, the neural network model is a U-NET-based model including an encoder and a decoder.

In an embodiment, the neural network model further includes an SE-block configured to generate a weight vector by comprising a 1D feature extracted from a convolutional layer of each layer in the encoder and concatenate the weight vector to input of a convolutional layer of each layer in the decoder by applying the weight vector to the 1D feature.

In an embodiment, the SE-block extracts a global feature including spatial information by squeezing the 1D and generates the weight vector composed of feature elements having a value between 0 and 1 by scaling the global feature.

In an embodiment, the SE-block concatenates the weight vector and the 1D feature to input of a convolutional layer of the same layer in the decoder by performing element-wise product on the weight vector and the 1D feature.

The present disclosure can increase accuracy in reconstruction of an ABP signal and blood pressure estimation by making the neural network model consider all of the morphological features and spatiotemporal features of ECG and PPG signals.

Detailed effects of the present disclosure in addition to the above effects will be described with the following detailed description for accomplishing the present disclosure.

### BRIEF DESCRIPTION OF THE DRAWINGS

The above and other objectives, features and other advantages of the present disclosure will be more clearly understood from the following detailed description when taken in conjunction with the accompanying drawings, in which:

FIG. 1 is a flowchart showing a reconstruction method of an arterial blood pressure (ABP) signal according to an embodiment of the present disclosure;

FIG. 2 is a diagram showing removing noises from ECG and PPG signals using a band pass filter;

FIG. 3 is a diagram for explaining a process of aligning ECG and PPG signals to generate an ECG-PPG signal;

FIG. 4 is a diagram showing an example of an ECG-PPG signal obtained by combining ECG and PPG signals;

FIG. 5 is a diagram showing the case in which a noise has been applied to an ECG-PPG signal at each of SNRs;

FIG. 6 is a diagram for explaining the operation of a neural network model of the present disclosure;

FIG. 7 is a flowchart showing a method of training a neural network model for blood estimation in addition to reconstruction of an ABP signal;

FIG. 8 is a diagram showing the structure of a neural network model according to an embodiment of the present disclosure;

FIG. 9 is a diagram for explaining a process of extracting features from an ECG-PPG signal on the basis of a weight included in an ECG-PPG signal;

FIG. 10 is diagram a comparing ABP signal reconstruction performance of a neural network model according to an embodiment of the present disclosure; and

FIG. 11 is a diagram comparing blood pressure estimation performance of a neural network model according to an embodiment of the present disclosure.

### DETAILED DESCRIPTION

The objects, characteristics, and advantages will be described in detail below with reference to the accompanying drawings, so those skilled in the art may easily achieve the spirit of the present disclosure. However, in describing the present disclosure, detailed descriptions of well-known technologies will be omitted so as not to obscure the description of the present disclosure with unnecessary details. Hereinafter, exemplary embodiments of the present disclosure will be described with reference to accompanying drawings. The same reference numerals are used to indicate the same or similar components in the drawings.

Although terms “first”, “second”, etc. are used to describe various components in the specification, it should be noted that these components are not limited by the terms. These terms are used to discriminate one component from another component and it is apparent that a first component may be a second component unless specifically stated otherwise.

Further, when a certain configuration is disposed “over (or under)” or “on (beneath)” a component in the specification, it may mean not only that the certain configuration is disposed on the top (or bottom) of the component, but that another configuration may be interposed between the component and the certain configuration disposed on (or beneath) the component.

Further, when a certain component is “connected”, “coupled”, or “jointed” to another component in the specification, it should be understood that the components may be directly connected or jointed to each other, but another component may be “interposed” between the components or the components may be “connected”, “coupled”, or “jointed” through another component.

Further, singular forms that are used in this specification are intended to include plural forms unless the context clearly indicates otherwise. In the specification, terms “configured”, “include”, or the like should not be construed as necessarily including several components or several steps described herein, in which some of the components or steps may not be included or additional components or steps may be further included.

Further, the term “A and/or B” stated in the specification means that A, B, or A and B unless specifically stated otherwise, and the term “C to D” means that C or more and D or less unless specifically stated otherwise.

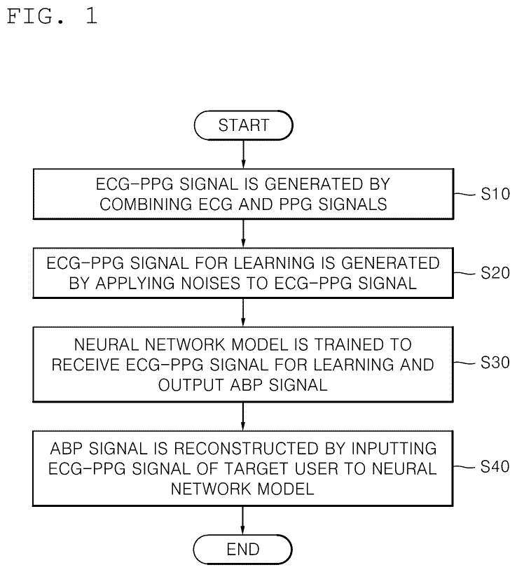

The present disclosure relates to a method of reconstructing an arterial blood pressure (ABP) corresponding to the morphological feature of a combination signal of ECC and PPG on the basis of the morphological feature. Hereafter, a reconstruction method of an arterial blood pressure (ABP) signal according to an embodiment of the present disclosure is described in detail with reference to FIGS. 1 to 11.

FIG. 1 is a flowchart showing a reconstruction method of an arterial blood pressure (ABP) signal according to an embodiment of the present disclosure.

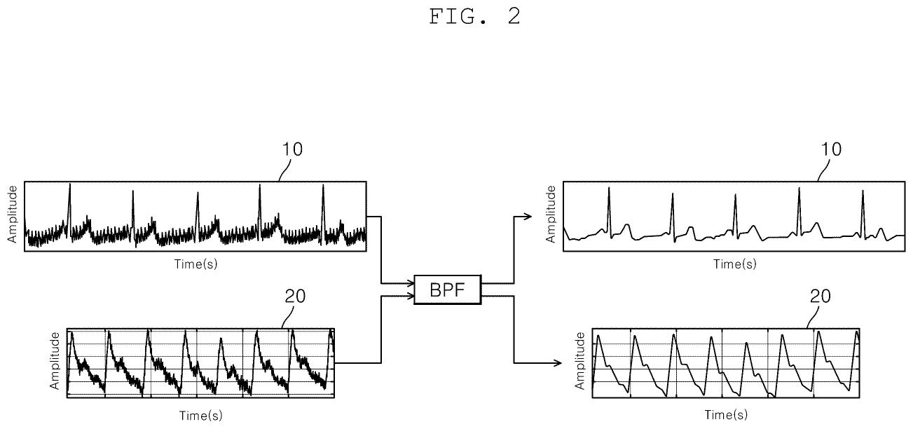

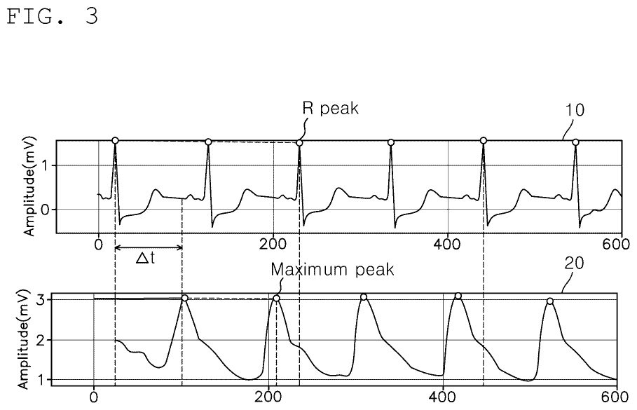

FIG. 2 is a diagram showing removing noises from ECG and PPG signals using a band pass filter. FIG. 3 is a diagram for explaining a process of aligning ECG and PPG signals to generate an ECG-PPG signal.

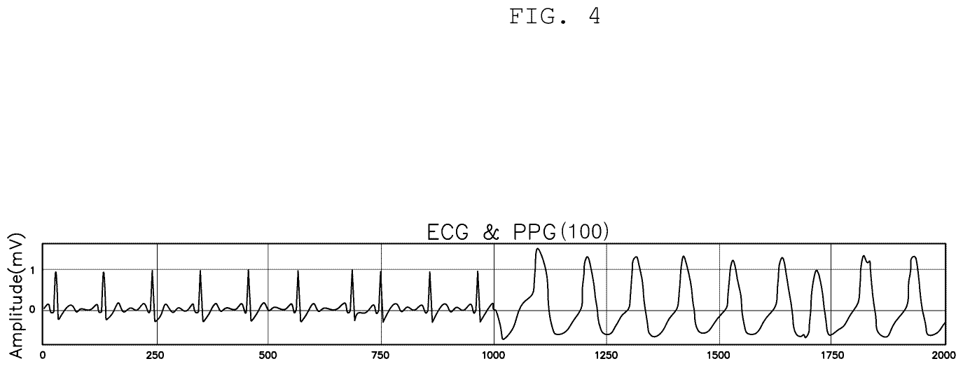

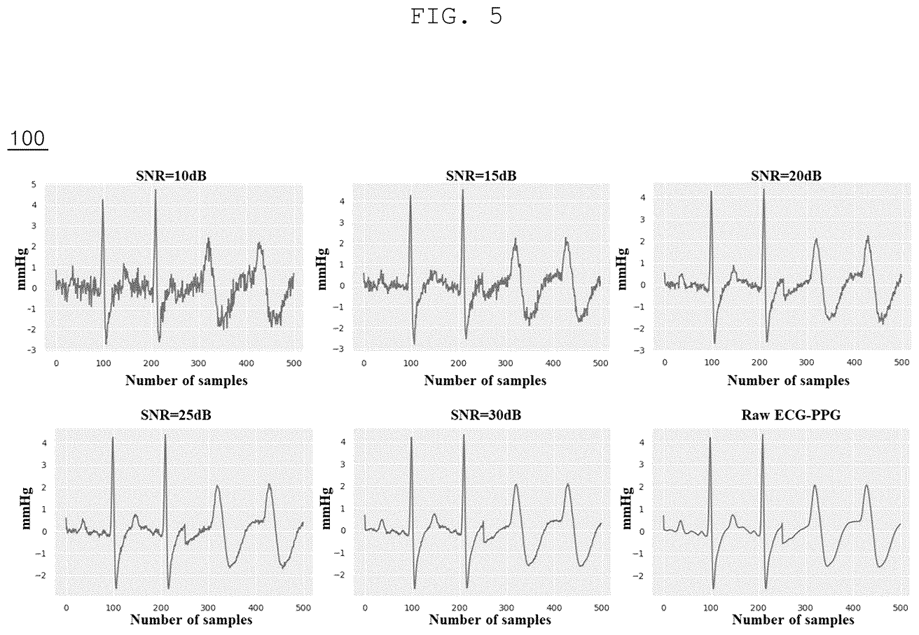

FIG. 4 is a diagram showing an example of an ECG-PPG signal obtained by combining ECG and PPG signals and FIG. 5 is a diagram showing the case in which a noise has been applied to an ECG-PPG signal at each of SNRs.

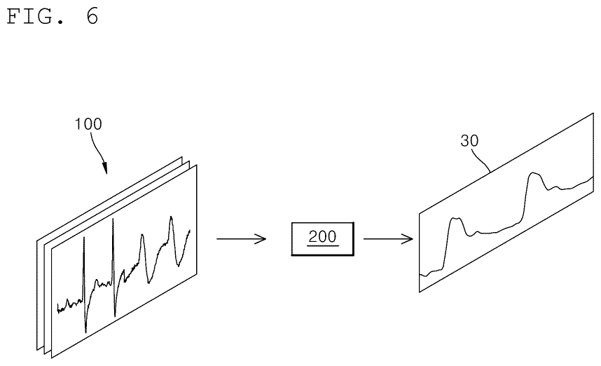

FIG. 6 is a diagram for explaining the operation of a neural network model of the present disclosure and

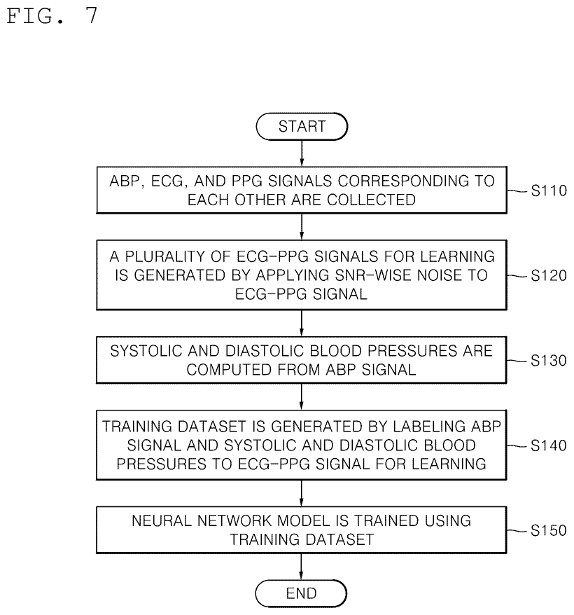

FIG. 7 is a flowchart showing a method of training a neural network model for blood estimation in addition to reconstruction of an ABP signal.

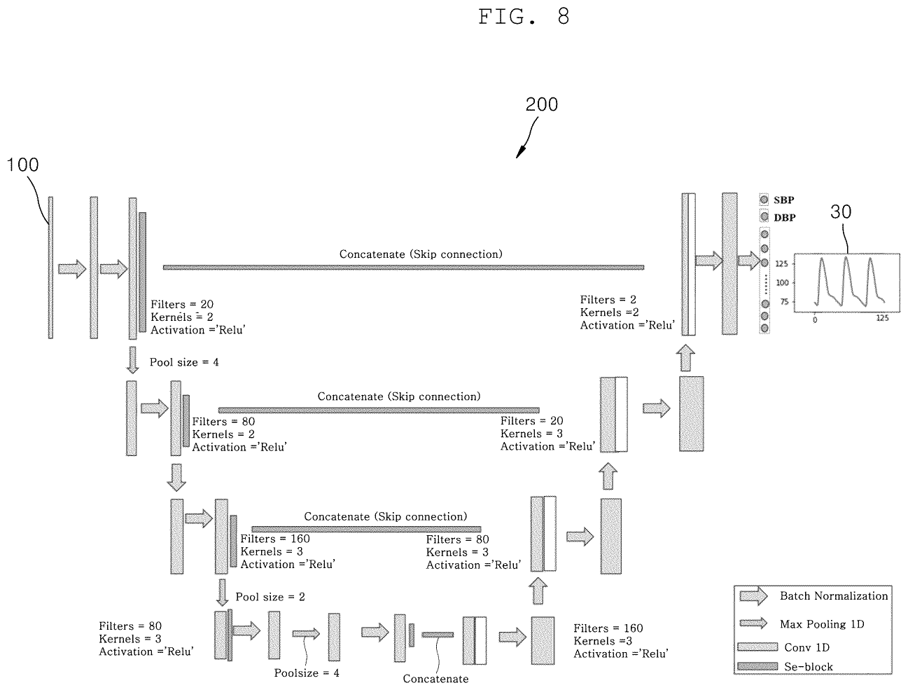

FIG. 8 is a diagram showing the structure of a neural network model according to an embodiment of the present disclosure.

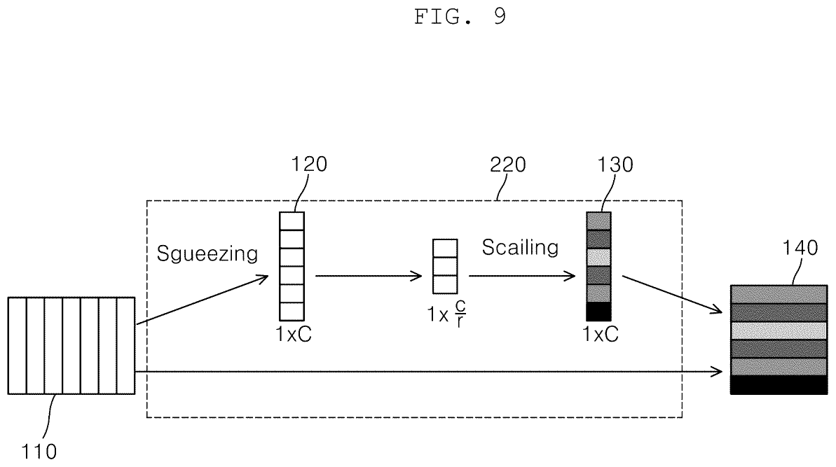

FIG. 9 is a diagram for explaining a process of extracting features from an ECG-PPG signal on the basis of a weight included in an ECG-PPG signal.

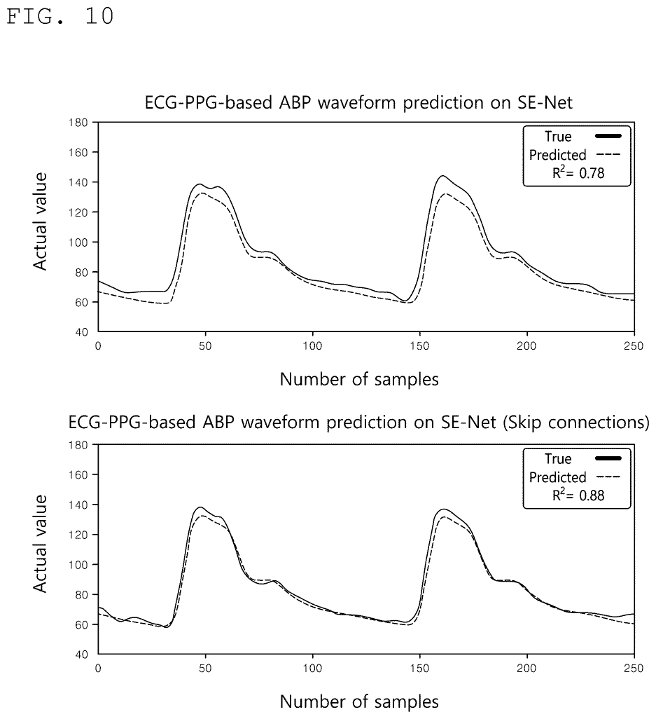

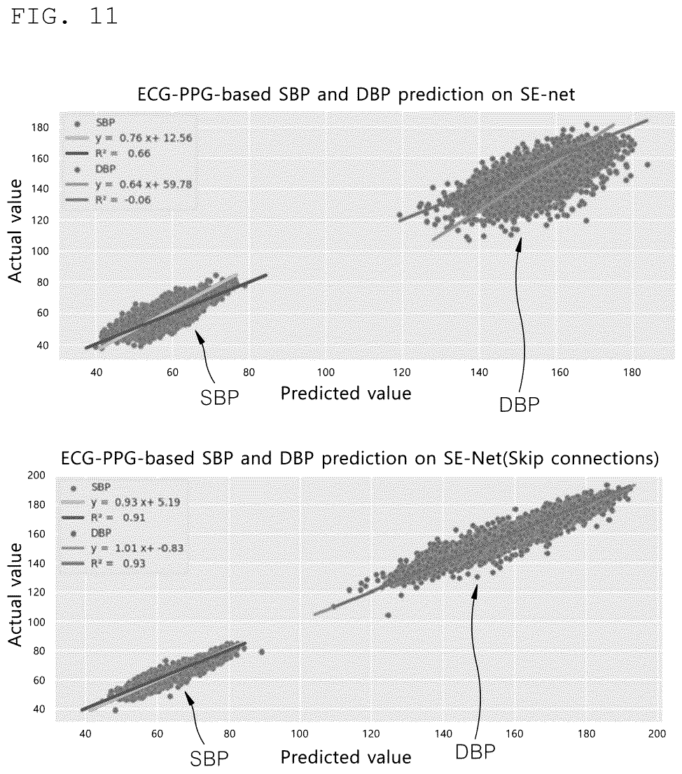

FIG. 10 is a diagram comparing ABP signal reconstruction performance of a neural network model according to an embodiment of the present disclosure and FIG. 11 is a diagram comparing blood pressure estimation performance of a neural network model according to an embodiment of the present disclosure.

Referring to FIG. 1, a reconstruction method of an arterial blood pressure (ABP) signal according to an embodiment of the present disclosure may include a step of generating an ECG-PPG signal by combining ECG and PPG signals (S10), a step of generating an ECG-PPG signal for learning by applying a noise to the ECG-PPG signal (S20), a step of training a neural network model to receive the ECG-PPG signal for learning and output an ABP signal (S30), and a step of reconstructing an APB signal by inputting an ECG-PPG signal of a target user into the neural network model (S40).

However, the reconstruction method of an ABP signal shown in FIG. 1 is based on an embodiment, the steps of the present disclosure are not limited to the embodiment shown in FIG. 1, and if necessary, some steps may be added, changed, or removed.

Meanwhile, the steps shown in FIG. 1 may be performed by a processor, and to this end, the processor may include at least one physical element of application specific integrated circuits (ASICs), digital signal processors (DSPs), digital signal processing devices (DSPDs), programmable logic devices (PLDs), field programmable gate arrays (FPGAs), micro-controllers, and a controller.

Hereafter, the steps shown in FIG. 1 are described in detail.

The processor can generate an ECG-PPG signal by combining ECG and PPG signals corresponding to an ABP signal (S10). In this case, the term “corresponding to each other” may mean that signals are acquired in the same time period. In detail, the ABP signal, the ECG signal, and the PPG signal may be signals measured from any one user in the same time period.

The processor can collect APB, ECG, and PPG signals corresponding to each other from various pre-constructed external databases. For example, the processor can collect APB, ECG, and PPG signals from a Medical Information Mart for Intensive Care (MIMIC) database storing biomedical signal data of patients collected at an intensive care unit.

The ECG signal, which is obtained by measuring an action current that is generated in the heart muscles in accompaniment with heartbeats, may include various items of information related to the state of a heart. In detail, the ECG signal may include a P-wave, a QRS-wave, and a T-wave and each of the waves may include information about depolarization of the atrial muscles, depolarization of the ventricular muscles, and repolarization. The ECG signal may be collected from a sensor including an electrode that comes in contact with a skin, and for example, may be collected through wireless communication with a wearable device equipped with an electrode.

Meanwhile, the PPG signal is obtained by measuring variation of the intensity of light emitted to a skin due to blood flow when the light is reflected and may include various items of information related to blood flow. In detail, the PPG signal may have periodicity that depends on heartbeats and intensity variation that depends on various nonlinear blood flow characteristics. The PPG signal may be collected from a certain sensor that can perform photoplethysmography, and for example, may be collected through wireless communication with a wearable device equipped with a photoplethysmographic sensor.

The processor can generate ECG-PPG signals for training the neural network model to be described below by combining an ECG signal and a PPG signal measured from many users in each of various time periods.

Meanwhile, the processor can remove noises included in an ECG signal and a PPG signal before generating an ECG-PPG signal.

Referring to FIG. 2, the processor according to an embodiment can remove noises by passing an ECG signal 10 and a PPG signal 20 through a band pass filter (BPF). In this case, a lower cutoff frequency that determines a pass band may be set as about 0.9 [Hz] so that low physiological vibrations related to breathing and blood vessel exercises can be conserved, and a higher cutoff frequency may be set as about 10 [Hz] to remove high-frequency noises.

Further, the processor can align an ECG signal 10 and a PPG signal 20 before generating an ECG-PPG signal.

Referring to FIG. 3, in an embodiment, the processor can detect an R peak in an ECG signal 10 and can detect a maximum peak in a PPG signal 20. In this case, the maximum peak may mean a peak of which the value is the maximum in one cycle of the PPG signal 20. Next, the processor can align the ECG signal 10 and the PPG signal 20 on the basis of a time offset ^{Δt} between the R peak and the maximum peak. Meanwhile, this alignment operation may be performed on ECG and PPG signals 10 and 20 in a preset unit time period rather than the entire ECG and PPG signals 10 and 20. For example, a unit time period is set as 6 seconds, as shown in FIG. 3, the processor can detect a plurality of (in detail, six) R peaks in an ECG signal 10 for 6 seconds and can detect a plurality of (in detail, five) maximum peaks in a PPG signal 20 for 6 seconds. Next, the processor can compute a time offset & between the R peak and the maximum peak of a preset order, for example, the first R peak and maximum peak of the plurality of R peaks and the plurality of maximum peaks.

The processor can align the ECG signal 10 and the PPG signal 20 by applying the time offset it to the ECG signal 10 or the PPG signal 20. In an embodiment, as shown in FIG. 3, when a time offset 4 is determined, the processor can align two signals on the basis of the ECG signal 10 by subtracting the time offset ^{Δt} from the PPG signal 20. On the contrary, the processor may align two signals on the basis of the PPG signal 20 by adding the time offset ^{Δt} to the ECG signal 10.

Next, the processor can generate an ECG-PPG signal 100 by combining the aligned ECG signal 10 and PPG signal 20.

In this case, combining two signals may mean combining signals measured in two different domains such that the signals are continuous in terms of time rather than combining the intensity of two signals.

In detail, referring to FIG. 4, the processor can generate an ECG-PPG signal 100 by time-serially connecting an ECG signal 10 and a PPG signal 20 aligned in a unit time period of 10 seconds. In other words, the processor can generate an ECG-PPG 100 by connecting the start point of a PPG signal 20 having a length of 10 seconds to the end of an ECG signal 10 having a length of 10 seconds.

Meanwhile, an ECG signal 10 and a PPG signal 20 may have intensity of different scales, and in the present disclosure, normalization or scaling may not be performed on the intensity of two signals so that a neural network model 200 to be described below can learn morphological differences between an ECG signal 10 and a PPG signal 20. The processor can generate a plurality of ECG-PPG signals 100 for learning by applying a plurality of noises, which is different in intensity, to each ECG-PPG signal 100 generated in step S10 (S20). In other words, the processor can generate n ECG-PPG signals 100 for learning by applying n noises, which are different in intensity, to each ECG-PPG signal 100. Accordingly, when previously generated ECG-PPG signals 100 are m and the number of noises that are different in intensity is n, m×n ECG-PPG signals 100 can be generated.

In detail, the processor can generate a plurality of ECG-PPG signals 100 for learning by applying each of noises having a plurality of preset Signal-to-Noise Ratios (SNRs) to ECG-PPG signals 100.

Referring to FIG. 5, in an embodiment, an ECG-PPG signal 100 may have the number of a total of 500 sample data, in which the front 250 pieces may show an ECG signal 10 and the rear 250 pieces may show a PPG signal 20. The processor can apply different SNRs defined by the following [Equation 1], for example, white Gaussian noises of 10 dB, 15 dB, 20 dB, 25 dB, and 30 dB to an ECG-PPG signal 100, and accordingly, one ECG-PPG signal 100 may be reconstructed into five ECG-PPG signals 100 for learning,

$$S N R = 10 {log}_{10} \frac{Raw\_signal}{P noise} \qquad [ Equation 1 ]$$

(where Raw_signal is the average intensity of the ECG-PPG signal 100 and P_noise is a noise).

The processor can train the neural network model 200 to receive an ECG-PPG signal 100 for learning augmented in accordance with the method described above and output an ABP signal (S30).

Referring to FIG. 6, the neural network model 200 according to an embodiment of the present disclosure can be trained to receive ECG-PPG signals 100 of which the number has been augmented due to application of noises and output an ABP signal 300 corresponding to ECG and PPG signals 10 and 20. To this end, the processor can apply supervised learning to the neural network model 200 by setting the ECG-PPG signal 100 for learning as input data of the neural network model 200 and labeling the ABP signal 30 as output data of the neural network model 200.

In detail, the processor can input ECG-PPG signals 100 for learning into the neural network model 200 and apply supervised learning to the neural network model 200 such that the difference between an predicted signal output from the neural network model 200 and the labeled ABP signal 300 becomes minimum. To this end, the processor can set a loss function proportioned to |predicted signal−labeled ABP signal (30)| and can train the neural network model 200 such that the loss function becomes minimum.

The neural network model 200 can learn the correlation between the ECG-PPG signals 100 for learning and the ABP signal 30 through the process described above, and then can receive ECG-PPG signals 100 not used in learning and output an ABP signal 30 corresponding thereto. Meanwhile, the neural network model 200 that is applied to the present disclosure may have a certain structure that can learn the correlation between input and output data, and for example, may be implemented as a U-NET-based model including an encoder and a decoder.

The processor can reconstruct an ABP signal of a target user by inputting an ECG-PPG signal 100 of the target user into the neural network model 200 trained in accordance with step S30. As described above, since the neural network model 200 has already learned the correlation between ECG-PPG signals 100 and the ABP signal 30, the neural network model 200 can receive an ECG-PPG signal 100 of a target user not used in learning and predict an ABP signal 30 of the target user.

Meanwhile, the processor can train the neural network model 200 such that the neural network model 200 can output even a Systolic Blood Pressure (SBP) and a Diastolic Blood Pressure (DBP) of a target user in addition to an ABP signal 30, and hereafter, the operation of the present disclosure is described on the basis of an implemented example of the neural network model 200.

Referring to FIG. 7, a method of training the neural network model 200 according to an embodiment of the present disclosure may include a step of collecting an ABP signal and ECG and PPG signals 10 and 20 corresponding to each other (S110), a step of generating a plurality of ECG-PPG signals 100 for learning by applying an SNR-wise noise to an ECG-PPG signal 100 (S120), a step of computing systolic and diastolic blood pressures from an ABP signal 30 (S130), a step of generating a training dataset by labeling the ABP signal 30 and the systolic and diastolic blood pressures on the ECG-PPG signal 100 for learning (S140), and a step of training the neural network model 200 using the training dataset (S150).

However, the method of training the neural network model 200 shown in FIG. 7 is based on an embodiment, the steps of the present disclosure are not limited to the embodiment shown in FIG. 7, and if necessary, some steps may be added, changed, or removed.

Hereafter, the steps shown in FIG. 7 are described in detail and detailed description of the same matters as the operations of step S10 to step S40 is omitted.

The processor can collect an ABP signal and ECG and PPG signals 10 and 20 corresponding to each other from various pre-constructed external databases (S110) and can generate an ECG-PPG signal 100 for learning in accordance with step S10 and step S20 described above (S120).

Meanwhile, the processor can compute systolic and diastolic blood pressures from the ABP signal 30 collected in step S110 (S130). The processor can compute systolic and diastolic blood pressures from the ABP signal 30 through various methodologies known in the art, and, in an embodiment, the processor can compute a high peak as a systolic blood pressure and a low peak as a diastolic blood pressure.

Next, the processor can generate a training dataset by labeling the APB signal 30 and the systolic and diastolic blood pressures on the ECG-PPG signal 100 for learning (S140). In other words, the processor can generate a training data including input data: [ECG-PPG signal (100) for learning] and output data: [ABP signal (30), systolic blood pressure, diastolic blood pressure] by setting the ABP signal 30 and the systolic and diastolic blood pressures as labels (Ground Truth (GT)) for the ECG-PPG signal 100 for learning.

Next, the processor can apply supervised learning to the neural network model 200 such that the neural network model 200 receives the ECG-PPG signal 100 for learning and output an ABP signal 30 and systolic and diastolic blood pressures. In detail, the processor can input the ECG-PPG signal 100 for learning into the neural network model 200 and train the neural network model 200 such that the difference between predicted values output from the neural network model 200, that is, signal, [predicted ABP predicted systolic blood pressure, and predicted diastolic blood pressure] and the labeled [ABP signal 30, systolic blood pressure, and diastolic blood pressure] becomes minimum.

To this end, the processor can set a first loss function proportioned to |predicted ABP signal−labeled ABP signal (30)|, a second loss function proportioned to |predicted systolic blood pressure-labeled systolic blood pressure|, and a third loss function proportioned to |predicted diastolic blood pressure−labeled diastolic blood pressure|, and can train the neural network model 200 such that the first to third loss functions each become minimum.

Meanwhile, referring to FIG. 8, the neural network model may be implemented as a U-NET-based model including an encoder and a decoder. In this case, the neural network model 200 may further include an SE-block 220 that extracts a 1D feature from output of a convolutional layer of each layer in the encoder, generates a weight vector by compressing the extracted 1D feature, and combines the weight vector with input of convolutional layers of the same layers in the decoder by applying the weight vector to the 1D feature.

As shown in FIG. 8, the processor can extract a 1D feature by inputting an ECG-PPG signal 100 for learning into a 1D convolutional layer. Since the ECG-PPG signal 100 for learning is defined as intensity over time, it may be a 1D signal that is expressed as the number of data sampled over time. The convolutional layer of each layer included in the encoder of the neural network model 200 can extract a 1D feature including temporal information by time-serially convoluting a 1D kernel to a 1D signal that is input to itself.

The SE-block 220 can generate a weight vector by compressing the 1D feature extracted from the convolutional layer of each layer in the encoder. In detail, the 1D feature includes a plurality of feature values and the SE-block 220 can generate a weight vector showing which feature values of the plurality of feature values are more important parts in ABP signal reconstruction and blood pressure estimation.

Hereafter, the operation of the SE-block 220 is described in more detail with reference to FIG. 9.

Referring to FIG. 9, the SE-block 220 can extract a global feature by squeezing a 1D feature 110. In an example, the SE-block 220 can extract a 1D global feature (1×c) 120 by applying pooling, in detail, global average pooling to the 1D feature 110. In this process, spatial information involved in the ECG-PPG signal 100 for learning may be included in the global feature 120.

Next, the SE-block can generate a weight vector 130 composed of elements having a value between 0 and 1 by recalibrating, in detail, contracting and expanding the global feature in accordance with a preset reduction ratio r for considering dependencies between values included in the global feature and by scaling the recalibrated global feature.

Next, the SE-block 220 can generate a weighted feature 140 by applying the weight vector 130 to the previously extracted 1D feature 110 and can concatenate the weighted feature 140 to the input of the convolutional layer of the same layer in the decoder. In detail, the SE-block 220 can generate the weighted feature 140 by performing element-wise product on the weight vector and the 1D feature and can concatenate the weighted feature 140 to the input of the convolutional layer of the same layer in the decoder.

Referring to FIG. 8 again, each SE-block 220 can connect an encoder and a decoder of the same layer through skip connection in a U-NET-based model. Accordingly, each SE-block 220 can apply the weight vector 130 to the 1D feature 110 extracted from the convolutional layer of each layer in the encoder and can concatenate the result value 140 to the input of the convolutional layer of the same layer in the decoder

Through this way, the neural network model 200 can reconstruct an ABP signal and determine systolic and diastolic blood pressures in consideration of all of the morphological feature, temporal feature, and spatial feature of an ECG-PPG signal 100 for learning.

When the neural network model 200 finishes being trained in the manner described above, the processor can input an ECG-PPG signal 100 of a target user into the neural network model 200. Since the neural network model 200 has learned the correlation between [ECG-PPG signals (100)] and [ABP signal (30), systolic blood pressure, and diastolic blood pressure], the neural network model 200 can receive an ECG-PPG signal 100 of a target user not used in learning and predict an ABP signal, a systolic blood pressure, and a diastolic blood pressure of the target user. The processor can reconstruct the ABP signal, for example, visualize a signal and can determine the systolic blood pressure and the diastolic blood pressure of the target user on the basis of output of the neural network model 200.

FIG. 10 shows comparison of the performance of reconstructing an ABP signal depending on whether an SE-block 220 is connected through skip connection using the U-NET-based neural network model 200 shown in FIG. 8. As shown in FIG. 10, it is possible to know that an R2 score (R squared score) is considerably higher in the case in which an SE-block 220 is connected in a skip connection type (R²=0.88) than another case in which it is not (R²=0.78).

FIG. 11 shows comparison of the performance of estimating a blood pressure depending on whether an SE-block 220 is connected through skip connection using the U-NET-based neural network model 200 shown in FIG. 8. As shown in FIG. 11, it is possible to know that an R2 score is considerably higher in the case in which an SE-block 220 is connected in a skip connection type (R²=0.91) than another case in which it is not (R²=0.66).

As described above, the present disclosure has the advantage that it is possible to increase accuracy in reconstruction of an ABP signal and blood pressure estimation by making the neural network model 200 consider all of the morphological features and spatiotemporal features of ECG and PPG signals 10 and 20.

Although the present disclosure was described with reference to the exemplary drawings, it is apparent that the present disclosure is not limited to the embodiments and drawings in the specification and may be modified in various ways by those skilled in the art within the range of the spirit of the present disclosure. Further, even though the operation effects according to the configuration of the present disclosure were not clearly described with the above description of embodiments of the present disclosure, it is apparent that effects that can be expected from the configuration should be also admitted.

What is claimed is:

1. A method of reconstructing an arterial blood pressure signal, the method comprising:
generating, by a processor, an ECG-PPG signal by combining ECG and PPG signals corresponding to an ABP signal;
generating, by a processor, a plurality of ECG-PPG signals for learning by applying a plurality of noises, which is different in intensity, to the ECG-PPG signal;
training, by a processor, a neural network model to receive the ECG-PPG signals for learning and output the ABP signal; and
reconstructing, by a processor, an ABP signal of a target user by inputting an ECG-PPG signal of the target user into the neural network model.

2. The method of claim 1, wherein the generating of an ECG-PPG signal includes:
removing noises in the ECG signal and the PPG signal by passing the ECG signal and the PPG signal through a band pass filter (BPF); and
generating the ECG-PPG signal by combining the ECG signal and PPG signal with the noises removed.

3. The method of claim 1, wherein the generating of an ECG-PPG signal includes:
detecting an R peak in the ECG signal;
detecting a maximum peak in the PPG signal;
aligning the ECG signal and the PPG signal on the basis of a time offset between the R peak and the maximum peak; and
generating the ECG-PPG signal by combining the aligned ECG signal and PPG signal.

4. The method of claim 1, wherein the generating of an ECG-PPG signal includes:
detecting a plurality of R peaks in an ECG signal in a unit time period;
detecting a plurality of maximum peaks in a PPG signal in the unit time period;
computing a time offset between an R peak and a maximum peak of a preset order of the plurality of R peaks and the plurality of maximum peaks;
aligning the ECG signal and the PPG signal by applying the time offset to the ECG signal or the PPG signal; and
generating the ECG-PPG signal by combining the aligned ECG signal and PPG signal.

5. The method of claim 1, wherein the generating of an ECG-PPG signal includes generating the ECG-PPG signal by connecting the ECG signal and the PPG signal such that the ECG signal and the PPG signal are temporally continuous.

6. The method of claim 1, wherein the generating of an ECG-PPG signal for learning includes generating a plurality of ECG-PPG signals for learning by applying each of noises having a plurality of preset Signal-to-Noise Ratios (SNRs) to the ECG-PPG signal.

7. The method of claim 6, wherein the SNR is defined by the following [Equation 1],
$$S N R = 10 {log}_{10} \frac{Raw\_signal}{P noise} \qquad [ Equation 1 ]$$
(where Raw_signal is average intensity of the ECG-PPG signal and P_noise is the noise).

8. The method of claim 1, wherein the training of a neural network model includes applying supervised learning to the neural network model by setting each of the ECG-PPG signals as input data of the neural network model and setting the APB signal as output data of the neural network model.

9. The method of claim 1, further comprising computing, by a processor, a systolic blood pressure and a diastolic blood pressure from the APB signal,
wherein the training of a neural network model includes training the neural network model such that the neural network model receives each of the ECG-PPG signals and outputs the ABP signal and the systolic and diastolic blood pressures, and
the reconstructing of an ABP signal includes determining an ABP signal and systolic and diastolic blood pressures of a target user by inputting an ECG-PPG signal of the user into the neural network model.

10. The method of claim 1, wherein the neural network model is a U-NET-based model including an encoder and a decoder.

11. The method of claim 10, wherein the neural network model further includes an SE-block configured to generate a weight vector by comprising a 1D feature extracted from a convolutional layer of each layer in the encoder and concatenate the weight vector to input of a convolutional layer of each layer in the decoder by applying the weight vector to the 1D feature.

12. The method of claim 11, wherein the SE-block extracts a global feature including spatial information by squeezing the 1D feature and generates the weight vector composed of elements having a value between 0 and 1 by scaling the global feature.

13. The method of claim 11, wherein the SE-block concatenates the weight vector and the 1D feature to input of a convolutional layer of the same layer in the decoder by performing element-wise product on the weight vector and the 1D feature.
